(19) United States
(12) Patent Application Publication
(10) Pub. No.: US 2025/0143034 A1
(43) Pub. Date: May 1, 2025

(54) OPTICAL ASSEMBLY WITH PHOSPHOR CONVERSION

(71) Applicant: Abiomed, Inc., Danvers, MA (US)

(72) Inventors: Claude Belleville, Danvers, MA (US); Andrew Gentile, Danvers, MA (US)

(73) Assignee: Abiomed, Inc., Danvers, MA (US)

(21) Appl. No.: 18/918,518

(22) Filed: Oct. 17, 2024

Related U.S. Application Data
(60) Provisional application No. 63/591,208, filed on Oct. 18, 2023.

(51) Int. Cl. H10H 20/851; G01L 1/24

(52) U.S. Cl. CPC H10H 20/8513; G01L 1/242

(57) ABSTRACT

A dual-band phosphor-converted light emitting diode (PC-LED) is provided. The dual-band PC-LED includes a blue LED configured to generate blue light, a first phosphor-based light filter configured to output visible light based on the blue light; and a second phosphor-based light filter configured to output near-infrared light based on the visible light.

### RELATED APPLICATIONS

This application claims the benefit under 35 U.S.C. § 119 (e) to U.S. Provisional Patent Application No. 63/591,208, filed Oct. 18, 2023, and titled, “OPTICAL ASSEMBLY WITH PHOSPHOR CONVERSION,” the entire contents of which is incorporated by reference herein.

### FIELD OF THE INVENTION

This disclosure relates to an optical assembly with phosphor conversion.

### BACKGROUND

A phosphor-converted light emitting diode (PC-LED) includes a blue, ultra-violet or electron beam light source in combination with a phosphor layer. The phosphor layer converts a light signal from the light source to a broadband signal of longer wavelength. For example, when a blue LED is used as the light source, the phosphor layer absorbs blue light from the light sources and emits visible white light.

PC-LEDs have primarily been developed for lighting applications in which a warm white light is desired, such as under-cabinet lighting for kitchen countertops. In a PC-LED, white light is generally obtained by converting a pumping blue signal from a blue LED to red, amber, yellow, or another longer wavelength signal. The perception of white light generated by the PC-LED is created by allowing a certain portion of the pumped blue light signal through the phosphor layer, where the two colors add to create the perception of white light.

### SUMMARY

Described herein are systems and methods for forming and/or using a phosphor-converted optical assembly.

In some embodiments, a dual-band phosphor-converted light emitting diode (PC-LED) is provided. The dual-band PC-LED includes a blue LED configured to generate blue light, a first phosphor-based light filter configured to output visible light based on the blue light, and a second phosphor-based light filter configured to output near-infrared light based on the visible light.

In one aspect, the first phosphor-based light filter is arranged between the blue LED and the second phosphor-based light filter. In another aspect, the second phosphor-based light filter comprises a phosphor mixture including phosphor and an adhesive. In another aspect, the adhesive comprises epoxy. In another aspect, the phosphor mixture includes at least 10% phosphor by volume. In another aspect, the phosphor mixture includes at least 30% phosphor by volume. In another aspect, the phosphor mixture includes at least 50% phosphor by volume. In another aspect, the first phosphor-based light filter is further configured to pass at least some of the blue light, and the second phosphor-based light filter is configured to output the near-infrared light further based on the blue light passed through the first phosphor-based light filter. In another aspect, the first phosphor-based light filter includes less than 30% phosphor by volume. In another aspect, the first phosphor-based light filter includes less than 10% phosphor by volume. In another aspect, a first spectrum of visible light output by the first phosphor-based light filter has a peak wavelength in a range of 550-600 nm. In another aspect, the first spectrum has a peak wavelength in the range of 590-610 nm. In another aspect, a second spectrum of near-infrared light output by the second phosphor-based light filter has a peak wavelength in a range of 800-900 nm. In another aspect, the second spectrum has a peak wavelength in a range of 820-850 nm. In another aspect, a first spectrum of visible light output by the first phosphor-based light filter has a first peak wavelength, a second spectrum of near-infrared light output by the second phosphor-based light filter has a second peak wavelength, and the first peak wavelength is ⅘ of the second peak wavelength.

In some embodiments, an optical pressure sensor system is provided. The optical pressure sensor system includes a dual-band phosphor-converted light emitting diode (PC-LED). The dual-band PC-LED is configured to output light having a mixture of visible light and near-infrared light. The optical pressure sensor further includes at least one sensor coupled to the dual-band PC-LED via one or more optical fibers, a detector module coupled to the at least one sensor. The detector module includes at least one lens, and an image sensor configured to sense light received from the at least one lens. The optical pressure sensor further includes at least one hardware processor configured to determine based, at least in part, on the light sensed by the image sensor, a pressure measured at the at least one sensor.

In one aspect, the dual-band PC-LED includes a blue LED configured to output blue light, a first phosphor-based light filter configured to output visible light based on the blue light, and a second phosphor-based light filter configured to output near-infrared light based on the visible light. In another aspect, the first phosphor-based light filter is arranged between the blue LED and the second phosphor-based light filter. In another aspect, the first phosphor-based light filter is further configured to pass at least some of the blue light, and the second phosphor-based light filter is configured to output the near-infrared light further based on the blue light passed through the first phosphor-based light filter. In another aspect, a first spectrum of the visible light has a peak wavelength in a range of 550-600 nm. In another aspect, the first spectrum has a peak wavelength in the range of 590-610 nm. In another aspect, a second spectrum of the near-infrared light has a peak wavelength in a range of 800-900 nm. In another aspect, the second spectrum has a peak wavelength in a range of 820-850 nm. In another aspect, a first spectrum of the visible light has a first peak wavelength, a second spectrum of the near-infrared light has a second peak wavelength, and the first peak wavelength is ⅘ of the second peak wavelength. In another aspect, the at least one sensor comprises a plurality of sensors, each of which is coupled to the dual-band PC-LED via the one or more optical fibers.

In some embodiments, an optical fiber assembly is provided. The optical fiber assembly includes an optical fiber having a core and a cladding surrounding the core. The optical fiber assembly further includes a phosphor-based light filter arranged at an end of the optical fiber, the phosphor-based light filter in contact with the core and the cladding at the end of the optical fiber.

In one aspect, the optical fiber assembly further includes a ferrule, wherein the optical fiber is arranged within the ferrule, and the phosphor-based light filter is arranged on a surface of the ferrule. In one aspect, the optical fiber assembly further includes an optical connector, wherein the optical connector includes the ferrule.

In one aspect, the phosphor-based light filter comprises a phosphor mixture including phosphor and an adhesive. In one aspect, the adhesive comprises epoxy. In one aspect, the phosphor mixture includes at least 10% phosphor by volume. In one aspect, the phosphor mixture includes at least 30% phosphor by volume. In one aspect, the phosphor mixture includes at least 50% phosphor by volume.

In one aspect, the phosphor-based light filter is configured to absorb at least 90% of blue light when introduced to phosphor-based light filter. In one aspect, the phosphor-based light filter is configured to absorb at least 99% of blue light when introduced to phosphor-based light filter. In one aspect, a thickness of the phosphor-based light filter is less than 50 μm. In one aspect, a thickness of the phosphor-based light filter is less than 10 μm. In one aspect, the phosphor-based filter is configured to emit white light.

In some embodiments, an optical connector is provided. The optical connector includes a ferrule having an optical fiber disposed therein. The optical fiber includes a core, and a cladding surrounding the core. The optical connector further includes a phosphor-based light filter arranged in contact with an end of the ferrule, and configured to condition light entering the ferrule.

In one aspect, the phosphor-based light filter comprises a phosphor mixture including phosphor and an adhesive. In one aspect, the adhesive comprises epoxy. In one aspect, the phosphor mixture includes at least 10% phosphor by volume. In one aspect, the phosphor mixture includes at least 30% phosphor by volume. In one aspect, the phosphor mixture includes at least 50% phosphor by volume.

In one aspect, the phosphor-based light filter is configured to absorb at least 90% of blue light when introduced to phosphor-based light filter. In one aspect, the phosphor-based light filter is configured to absorb at least 99% of blue light when introduced to phosphor-based light filter. In one aspect, a thickness of the phosphor-based light filter is less than 50 μm. In one aspect, a thickness of the phosphor-based light filter is less than 10 μm. In one aspect, the phosphor-based filter is configured to emit white light.

In some embodiments, a phosphor-converted light emitting diode (PC-LED) is provided. The PC-LED includes an optical fiber that includes a core and a cladding surrounding the core. The PC-LED further includes a phosphor-based light filter arranged at an end of the optical fiber, the phosphor-based light filter in contact with the core and the cladding at the end of the optical fiber, and an LED having an emission surface coupled to the phosphor-based light filter such that light generated by the LED is conditioned by the phosphor-based light filter prior to be transmitted through the optical fiber.

In one aspect, the phosphor-based light filter comprises a phosphor mixture including phosphor and an adhesive. In one aspect, the adhesive comprises epoxy. In one aspect, the phosphor mixture includes at least 10% phosphor by volume. In one aspect, the phosphor mixture includes at least 30% phosphor by volume. In one aspect, the phosphor mixture includes at least 50% phosphor by volume.

In one aspect, the LED comprises a blue LED configured to emit blue light into the phosphor-based light filter. In one aspect, the phosphor-based light filter is configured to absorb at least 90% of the blue light emitted into the phosphor-based light filter. In one aspect, the phosphor-based light filter is configured to absorb at least 99% of the blue light emitted into the phosphor-based light filter. In one aspect, the phosphor-based filter is configured to mix at least a portion of the blue light emitted into the phosphor-based light filter and longer wavelength light to generate white light. In one aspect, the longer wavelength light is amber light. In one aspect, a thickness of the phosphor-based light filter is less than 50 μm. In one aspect, a thickness of the phosphor-based light filter is less than 10 μm.

In some embodiments, an optical pressure sensor is provided. The optical pressure sensor includes a light emitting diode (LED), a sensor coupled to the LED via an optical fiber, the optical fiber including a phosphor-based light filter arranged at an end of the optical fiber, the phosphor-based light filter arranged to condition light emitted by the LED, a detector module coupled to the sensor, and at least one hardware processor. The detector module includes at least one lens, and an image sensor configured to sense light received from the at least one lens. The at least one hardware processor is configured to determine based, at least in part, on the light sensed by the image sensor, a pressure measured at the sensor.

In one aspect, the LED is a blue LED configured to emit blue light. In one aspect, a spectrum of light emitted by the phosphor-based light filter has a peak wavelength in a range of 550-600 nm. In one aspect, the spectrum has a peak wavelength in the range of 590-610 nm.

### BRIEF DESCRIPTION OF DRAWINGS

FIG. 1A schematically illustrates a first implementation of a phosphor-converted light emitting diode (PC-LED).

FIG. 1B schematically illustrates a second implementation of a PC-LED.

FIG. 1C schematically illustrates the PC-LED of FIG. 1B arranged in contact with an optical fiber assembly.

FIG. 2 schematically illustrates a dual-band PC-LED, in accordance with some embodiments of the present technology.

FIG. 3 is a flowchart of a process for fabricating a dual-band PC-LED, in accordance with some embodiments of the present technology.

FIG. 4A schematically illustrates an optical fiber assembly including a phosphor-based filter, in accordance with some embodiments of the present technology.

FIGS. 4B-4E illustrate optical fiber assemblies with phosphor-converted filters, in accordance with some embodiments of the present technology.

FIG. 5 illustrates components of an optical pressure sensor system that includes a PC-LED in accordance with some embodiments of the present technology.

### DETAILED DESCRIPTION

Some embodiments of the present disclosure relate to methods and apparatus for fabricating and using a phosphor-converted (PC) optical assembly. The inventors have recognized and appreciated that conventional (e.g., commercially available) PC-LEDs do not have suitable characteristics for use in optical pressure sensors (e.g., an optical pressure sensor for an intracardiac heart pump system). For example, some conventional PC-LEDs configured to generate white light have spectral characteristics that differ substantially from the broadband spectral characteristics of light produced by, for example, a tungsten lamp. Although light output from multiple LEDs having different spectral characteristics (e.g., different peak wavelength frequencies) may be combined to approximate a broadband white light source, such a configuration adds complexity to an optical pressure sensor system. Some embodiments of the present disclosure relate to a dual-band PC-LED, configured to output light having spectral characteristics that approximate a broadband white light source, which may obviate the need to combine the outputs from multiple LEDs.

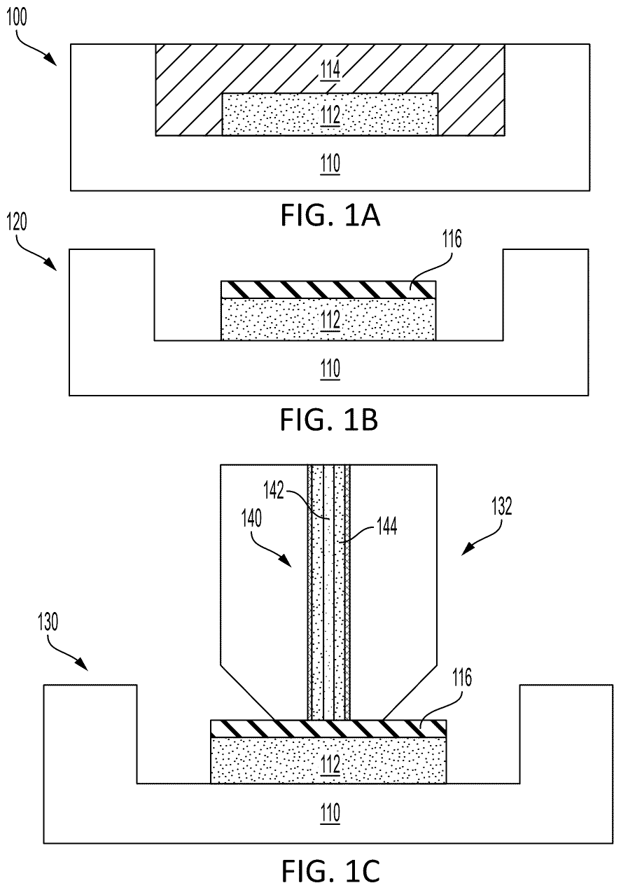

FIGS. 1A and 1B illustrate two conventional PC optical assemblies. FIG. 1A illustrates an optical assembly in which a blue LED 112 is arranged on a top surface of a receptacle 110. The receptacle 110 is then filled with a mixture 114 of phosphor powder and adhesive (e.g., silicone, epoxy, etc.). FIG. 1B illustrates an optical assembly 120 in which a blue LED 112 is also arranged on the top surface of a receptacle 110. However, rather than filling the receptacle 110 with the phosphor/adhesive mixture as in the optical assembly 100, in the optical assembly 120, a phosphor layer 116 including a mixture of phosphor powder and adhesive is deposited on the surface of the blue LED 112. When operational, the PC optical assemblies shown in FIGS. 1A and 1B may produce visible (white) light by filtering at least some of the blue light from the blue LED to output a light signal with a broader spectrum than the light provided by the blue LED.

FIG. 1C illustrates an optical assembly 130 coupled to an optical connector 132. Optical connector 132 may be implemented as a ferrule that includes an optical fiber 140 arranged therein. The optical fiber 140 may include a core 142 and a cladding 144 surrounding the core 142. In the example shown in FIG. 1C, optical assembly 130 is configured similarly to optical assembly 120 shown in FIG. 1B. For example, optical assembly 130 includes a phosphor layer 116, which is a mixture of phosphor powder and adhesive deposited on the surface of the blue LED 112. The blue LED 112 is arranged on a top surface of a receptacle 110. FIG. 1C shows the optical connector 132 including an end of the optical fiber 140 being arranged in contact with the phosphor layer 116 of the optical assembly 130. During operation, blue light generated from the blue LED 112 is passed through phosphor layer 116, which absorbs at least some (e.g., all) of the blue light and emits a broadband light signal of longer wavelength (e.g., amber light having a central wavelength between 600-650 nm). A portion of the light emitted from the phosphor layer 116, which includes a mixture (if any) of blue light not absorbed by phosphor layer 116 and longer wavelength light (e.g., amber light) is provided as input to the optical fiber 140 at the point of contact between the optical connector 132 and the phosphor layer 116 of the optical assembly 130.

As described herein the inventors have recognized that some conventional (e.g., commercially-available) PC-LEDs, such as those shown in FIGS. 1A and 1B do not generate light with suitable spectral characteristics for use in optical pressure sensors or other devices. For instance, such conventional PC-LEDs may produce light output that has a narrower spectral bandwidth than what would be desired to approximate a broadband white light source, such as a tungsten lamp. Some embodiments of the present disclosure relate to a multi-band PC-LED configured to output light having a broader spectrum than conventional PC-LEDs.

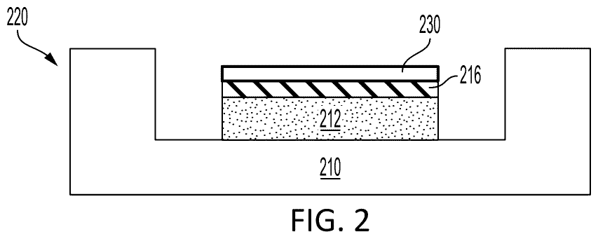

FIG. 2 schematically illustrates a structure of an example dual-band PC-LED in accordance with some embodiments of the present disclosure. FIG. 2 illustrates an optical assembly 220 in which a blue LED 212 is arranged on the top surface of a receptacle 210. A first phosphor-based layer 216 including a mixture of phosphor powder and adhesive may be formed on the surface of the blue LED 212. The first phosphor-based layer 216 may include a phosphor mixture that filters blue light from blue LED 212 to output visible (white) light. Optical assembly 220 further includes a second phosphor-based layer 230 including a mixture of phosphor powder and adhesive formed on the surface of the first phosphor-based layer 216. In some embodiments, the mixture of phosphor powder and adhesive in the second phosphor-based layer 230 may function to output light having spectral characteristics of near-infrared light. In this way, the two-layer phosphor-based structure shown in FIG. 2 may filter the blue light output from blue LED 212 to generate light having a mixture of visible (white) light and near-infrared light. In some embodiments, the combined light may have spectral characteristics that more closely approximate a broadband white light source, such as a tungsten lamp.

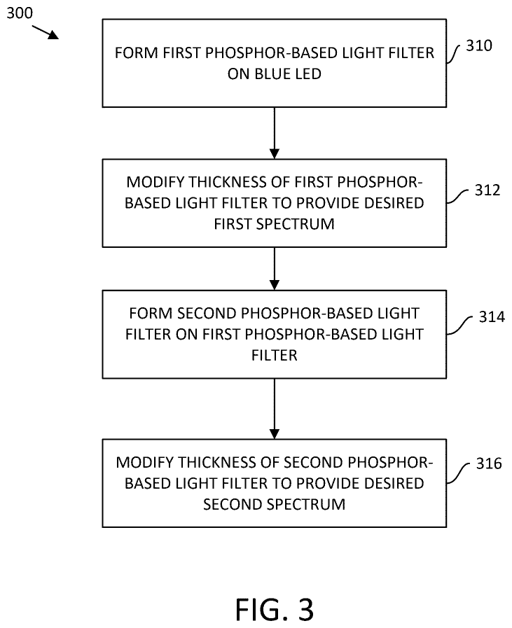

FIG. 3 is a flowchart of a process 300 for fabricating a dual-band PC-LED in accordance with some embodiments of the present disclosure. Process 300 may begin in act 310, where a first phosphor-based light filter (e.g., first phosphor-based layer 216) is formed on the surface of a blue LED. Process 300 may then proceed to act 312, where the thickness of the first phosphor-based light filter may be modified to tune the spectral properties of the light output from the first phosphor-based light filter. It should be appreciated that some embodiments of the present disclosure may omit acts 310 and 312 of process 300, and a conventional PC-LED that includes a blue LED and a phosphor-based layer may be used as a starting point create a dual-band PC-LED. Process 300 may then proceed to act 314, where a second phosphor-based light filter is formed on the first phosphor-based light filter. As described herein, the second phosphor-based light filter may have a different composition compared to the first phosphor-based light filter such that the second phosphor-based light filter converts light output from the first phosphor-based light filter (e.g., white light and any remaining blue light that was not absorbed by the first phosphor-based light filter) to light having a broader spectrum. For instance, the second phosphor-based light filter may include material that generates light having a near-infrared spectrum. When combined with the light output from the first phosphor-based light filter, the resulting light output from the dual-band PC-LED may have spectral properties desirable for use in an optical pressure sensor, as described herein. Process 300 may then proceed to act 316, where the thickness of second phosphor-based light filter may be modified to provide a desired spectrum of light output from the dual-band PC-LED. The thickness of the second phosphor-based light filter may be modified in any suitable way including, but not limited to, using an additive manufacturing approach to form thin layers of the material in second phosphor-based light filter on the surface of the first phosphor-based light filter until a desired thickness is reached, and using a technique to remove and/or spatially distribute material in the second phosphor-based light filter after deposition on the surface of the first phosphor-based light filter.

Conventional PC-LEDs are primarily designed for lighting applications, have a large surface of emission, and have an emission pattern similar to a block body emitter (i.e., emission in all directions). The inventors have recognized and appreciated that a light source with such a large emission surface and/or emission profile is not well suited for coupling to an optical fiber that has a much smaller diameter, as shown in FIG. 1C, because most of the emitted light is not coupled into the optical fiber, resulting in a low power transfer efficiency. Some embodiments of the present disclosure relate to techniques and optical assembly designs with increased power transfer efficiency when coupled to an optical fiber. Such designs may, for example, reduce the LED power consumption for the same amount of coupled light power.

In some embodiments of the present disclosure, the efficiency of the PC-LED device is increased by reducing the size of the blue pumping LED, such that more of the total emission surface of the LED is coupled to the end of the optical fiber. The inventors have recognized and appreciated, however, that when the emission surface of the LED is small, depositing a thin layer of a well-controlled mixture of phosphor and adhesive on the surface of the LED to create an optical assembly similar to that shown in FIGS. 1B and 1C may be challenging. For example, when the LED includes fragile electrical wires on its surface, there may be a risk that a needle used to deposit the phosphor mixture on the LED will disturb the fragile electrical wires. Alternatively, filling a receptacle of an LED optical assembly with a phosphor mixture as shown in FIG. 1A may not be efficient due to the inability to easily create a phosphor layer having a desired thickness optimal for high efficiency coupling into an optical fiber.

To remedy at least some of these disadvantages of existing techniques, some embodiments of the present disclosure relate to an optical fiber assembly that includes a phosphor-based light filter. By forming the phosphor-based light filter as part of an optical fiber assembly rather than as a layer formed as part of an optical assembly that includes the LED, at least some of the fabrication challenges described herein can be mitigated.

Additionally, the inventors have recognized that some conventional PC-LEDs are not tunable in the sense that the amount of blue light absorbed by the phosphor layer cannot be changed. Some embodiments of the technology described herein include a phosphor layer with a substantially larger percentage of phosphor compared to conventional PC-LEDs. The higher concentration of phosphor in the phosphor layer (e.g., >=30-50% phosphor by volume) enables the PC-LED to absorb more blue light from a blue LED source, resulting in an emitted light with a reduced blue light content. A PC LED configured to emit light with such characteristics may be useful, for example, as a light source for an optical pressure sensor as described herein.

In the examples provided herein, optical fiber assemblies that include a blue LED and a phosphor-conversion layer that emits amber light (also referred to herein as “PC amber LEDs”), while substantially blocking the emission of blue light are described. It should be appreciated however, that the techniques described herein may be used to fabricate optical fiber assemblies using other types of light sources (e.g., ultra-violet, electron beam) and phosphor conversion layers tuned to emit light with spectra other than amber. For example, the dual-band PC-LED structure described in connection with FIG. 2 may include multiple phosphor-conversion layers that generate visible (white) light with broadband characteristics suitable for use in an optical pressure sensor.

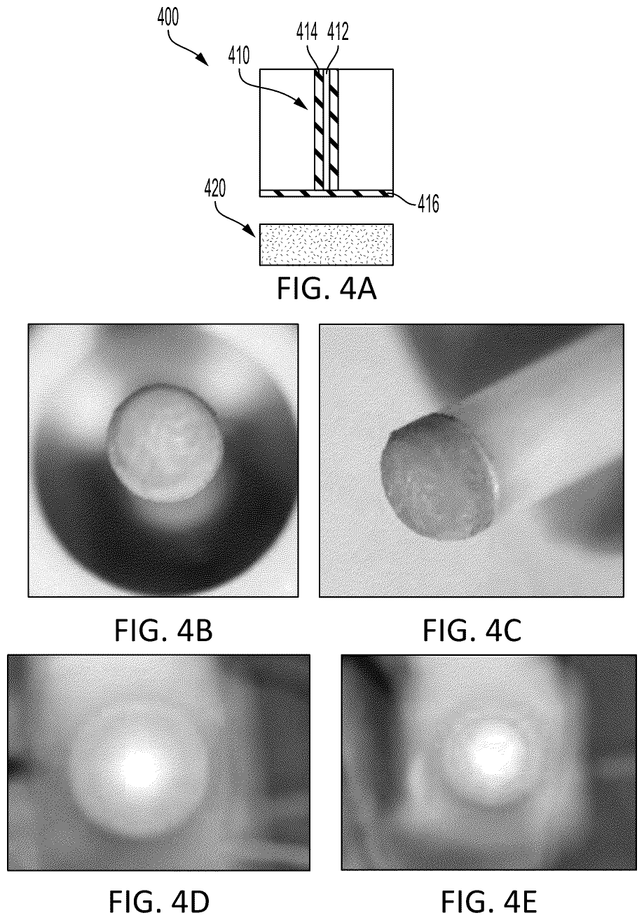

FIG. 4A schematically illustrates an optical fiber assembly 400 configured in accordance with some embodiments of the present disclosure. Optical fiber assembly 400 includes an optical fiber 410 having a core 412 and a cladding 414. In some embodiments, optical fiber assembly 400 includes a ferrule of an optical connector having arranged therein optical fiber 410. Optical fiber assembly 400 further includes a phosphor-based light filter 416 arranged at an end of the optical fiber 410. As shown in FIG. 4A, the phosphor-based light filter 416 is arranged to filter all light provided as input to optical fiber 410, such that any light launched into the optical fiber is conditioned by the phosphor mixture in the phosphor-based light filter 416. Although shown as being formed across the entire end of the optical fiber assembly 400, it should be appreciated that the phosphor-based light filter 416 may be formed in any suitable arrangement as long as the phosphor-based light filter 416 is arranged to condition light travelling through the optical fiber. For instance, in some embodiments, phosphor-based light filter 416 may be at least partially formed within the cladding/core of optical fiber 410 itself or may be formed at some other location of the optical fiber 410 rather than at the end of the optical fiber where light enters. For instance, in some embodiments, the phosphor-based light filter 416 may be formed at an end of the optical fiber where light exits the fiber.

The optical fiber assembly 400 having phosphor-based light filter 416 formed therein may then be coupled to a light source 420, such as a blue LED, configured to generate light incident on the optical fiber assembly 400. By forming the phosphor-based light filter 416 as part of the optical fiber rather than as part of an optical assembly that includes the light source, components (e.g., fragile electrical components) of the light source may be less susceptible to mechanical damage and/or a smaller amount of phosphor material may need to be used to create the phosphor-based light filter 416 (e.g., compared to the structure shown in FIG. 1A).

In some embodiments, an efficient PC-LED is fabricated taking into consideration one or more of the following:
the core of the optical fiber should be brought as close as possible to the surface of emission of the LED
the phosphor mixture should be thin to minimize the re-absorption and diffusion of converted light energy
the phosphor mixture should have a high concentration of phosphor (e.g., >30%, >50%).
the LED should be small, but should deliver a high density of energy (>μW/mm²)
assembly should be simple, repeatable, and controllable

In some embodiments, the phosphor mixture is formed to have a thickness that allows a desired amount of light at the emission frequency of a coupled LED to pass through the phosphor-based filter. For instance, when a blue LED is used as the coupled LED source, the thickness of the phosphor-based filter may be set such that no blue light passes through the phosphor-based filter, or such that some (e.g., 1%, 10%) of blue light passes through the phosphor-based filter. In some embodiments, the thickness of the phosphor-based light filter is less than 50 μm. In some embodiments, the thickness of the phosphor-based light filter is less than 10 μm. It should be appreciated that the phosphor-based filter shown in FIG. 4A may be formed using any suitable technique including preforming the phosphor-based filter to a desired thickness for a particular application if that thickness is known, prior to coupling of the filter with the optical fiber assembly.

FIGS. 4B-4E are photographs of an optical fiber assembly having a phosphor-based filter arranged on an end of the optical fiber assembly. FIGS. 4B and 4C show a phosphor-based filter arranged on an LC ferrule of an optical connector. FIGS. 4D and 4E show examples of light transmitted through a phosphor-based filter on an optical fiber assembly in accordance with the techniques described herein. The white appearance of the light transmitted through the phosphor-based filter indicates that at least some blue light is transmitted through the filter, with the transmitted light combining with amber light to generate warm white light.

An optical assembly including one or more phosphor-based light filters as described herein may be used in various applications in which such a tunable light source may be used. One such application is for use in an optical pressure sensor, such as a multi-channel optical pressure sensor. The inventors have recognized and appreciated that light sources for such sensors consume a large amount of the overall power budget for the sensor. Accordingly, to produce lower power consumption sensors, one or more conventional light sources (e.g., one or more conventional LED light sources) in an optical pressure sensor may be replaced with a PC-LED (e.g., a dual-band PC-LED, a PC-LED with increased concentration of phosphor material, or some combination thereof) as described herein.

In one particular implementation of a multi-channel optical pressure sensor for a circulatory support system (e.g., a heart pump) that may include two or more LED light sources, one or both of the multiple LED light sources may be replaced with a PC-LED as described herein to reduce the overall power consumption of the optical pressure sensor. For instance, a multi-channel optical pressure sensor may include two light emitting diodes (LEDs) configured to output light with different spectra, with the spectrum of light output from each of the two LEDs being selected such that their combined output has some characteristics similar to white light generated, for example, by a tungsten lamp. In some embodiments, the characteristics of the light emitted from a phosphor-based filter in a PC-LED may be selected to approximate the wavelength of light generated by a conventional LED source that the PC-LED is replacing. For instance, a multi-channel optical sensor may include a first light source configured to emit light having a peak wavelength in the range of 550-600 nm and a second light source configured to emit light having a peak wavelength in the range of 800-900 nm. In some embodiments of the present technology, the first light source may be replaced with a PC-LED source as described herein, wherein the phosphor-based filter is configured to emit amber light having a peak wavelength of approximately 600 nm. In other embodiments, both the first light source and the second light source may be replaced with a single dual-band PC LED source configured to output light having a complex spectrum with multiple peak wavelengths (e.g., a first peak wavelength in the range of 550-600 nm and a second peak wavelength in the range of 800-900 nm).

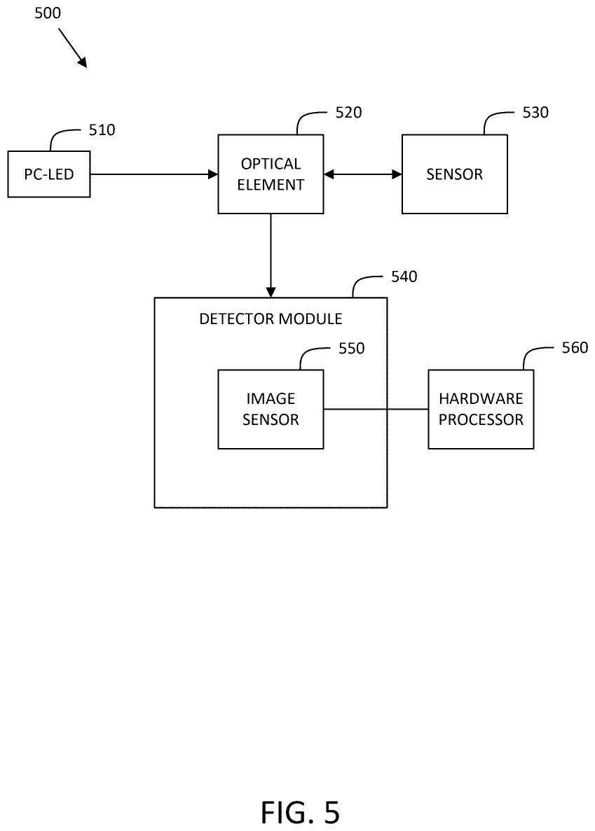

FIG. 5 schematically illustrates an optical pressure sensor system 500 designed in accordance with some embodiments of the present technology. System 500 includes PC-LED 510 and optical sensors 530 coupled via optical fibers and optical element 520. In the example system 500, PC-LED 510 may configured in accordance with the techniques described herein. For example, PC-LED 510 may be configured to output light having a spectrum that has some characteristics similar to white light generated, for example, by a tungsten lamp. In other embodiments, PC-LED 510 may be selected such that when combined with light having another spectrum (e.g., an LED configured to output light having a near-infrared spectrum), the combined output may have some characteristics similar to white light generated, for example, by a tungsten lamp. In some embodiments, the size of the PC-LED may be smaller than 1 millimeter. Such characteristics may facilitate the use of a lower-power LED light source to perform optical pressure sensing in a heart pump.

As shown, system 500 includes optical element 520 arranged between PC-LED 510 and sensor 530. In some embodiments, optical element 520 may be implemented as a splitter that provides light to sensor 530. Sensor 530 may be configured as a reflective element such that at least some of the light provided to sensor 530 is reflected back through optical element 520, which provides the reflected light as input to detector module 540. In this way, the reflected light signal provided by the sensor 530 may be further processed by components of detector module 540. As shown, detector module 540 may include an image sensor 550. System 500 may further include at least one hardware processor 560 configured to analyze signals captured by image sensor 550 to, for example, determine the pressure sensed by sensor 530. It should be appreciated that although only a single sensor 530 is shown in system 500, multiple sensors 530 may alternatively be used and light from PC-LED 510 may be routed using one or more optical fibers to each of the multiple sensors 530. In this way, an optical pressure sensing system may be configured to simultaneously sense pressure at multiple locations along a device (e.g., a heart pump) to which the optical pressure sensors are coupled.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1. A dual-band phosphor-converted light emitting diode (PC-LED), comprising:
a blue LED configured to generate blue light;
a first phosphor-based light filter configured to output visible light based on the blue light; and
a second phosphor-based light filter configured to output near-infrared light based on the visible light output from the first phosphor-based light filter.

2. The dual-band PC-LED of claim 1, wherein the first phosphor-based light filter is arranged between the blue LED and the second phosphor-based light filter.

3. The dual-band PC-LED of claim 1, wherein the second phosphor-based light filter comprises a phosphor mixture including phosphor and an adhesive.

4. (canceled)

5. The dual-band PC-LED of claim 3, wherein the phosphor mixture includes at least 10% phosphor by volume.

6. The dual-band PC-LED of claim 5, wherein the phosphor mixture includes at least 30% phosphor by volume.

7. The dual-band PC-LED of claim 6, wherein the phosphor mixture includes at least 50% phosphor by volume.

8. The dual-band PC-LED of claim 1, wherein
the first phosphor-based light filter is further configured to pass at least some of the blue light, and
the second phosphor-based light filter is configured to output the near-infrared light based on the blue light passed through the first phosphor-based light filter.

9. The dual-band PC-LED of claim 1, wherein the first phosphor-based light filter includes less than 30% phosphor by volume.

10. The dual-band PC-LED of claim 9, wherein the first phosphor-based light filter includes less than 10% phosphor by volume.

11. The dual-band PC-LED of claim 1, wherein a first spectrum of visible light output by the first phosphor-based light filter has a peak wavelength in a range of 550-600 nm.

12. (canceled)

13. The dual-band PC-LED of claim 11, wherein a second spectrum of the near-infrared light output by the second phosphor-based light filter includes a peak wavelength in a range of 800-900 nm.

14. (canceled)

15. The dual-band PC-LED of claim 1, wherein a first spectrum of visible light output by the first phosphor-based light filter has a first peak wavelength, a second spectrum of the near-infrared light output by the second phosphor-based light filter has a second peak wavelength, and the first peak wavelength is ⅘ of the second peak wavelength.

16. An optical pressure sensor system, comprising:
a dual-band phosphor-converted light emitting diode (PC-LED), wherein the dual-band PC-LED is configured to output light having a mixture of visible light and near-infrared light;
at least one sensor coupled to the dual-band PC-LED via one or more optical fibers;
a detector module coupled to the at least one sensor, the detector module comprising:
at least one lens; and
an image sensor configured to sense light received from the at least one lens; and
at least one hardware processor configured to determine based, at least in part, on the light sensed by the image sensor, a pressure measured at the at least one sensor.

17. The optical pressure sensor system of claim 16, wherein the dual-band PC-LED comprises:
a blue LED configured to output blue light;
a first phosphor-based light filter configured to output visible light based on the blue light; and
a second phosphor-based light filter configured to output near-infrared light based on the visible light output from the first phosphor-based light filter.

18-23. (canceled)

24. The optical pressure sensor system of claim 17, wherein a first spectrum of the visible light output by the first phosphor-based light filter has a first peak wavelength, a second spectrum of the near-infrared light output by the second phosphor-based light filter has a second peak wavelength, and the first peak wavelength is ⅘ of the second peak wavelength.

25. The optical pressure sensor system of claim 16, wherein the at least one sensor comprises a plurality of sensors, each of which is coupled to the dual-band PC-LED via the one or more optical fibers.

26-62. (canceled)

63. An optical pressure sensor, comprising:
a light emitting diode (LED);
a sensor coupled to the LED via an optical fiber, the optical fiber including a phosphor-based light filter arranged at an end of the optical fiber, the phosphor-based light filter arranged to condition light emitted by the LED;
a detector module coupled to the sensor, the detector module comprising:
at least one lens; and
an image sensor configured to sense light received from the at least one lens; and
at least one hardware processor configured to determine based, at least in part, on the light sensed by the image sensor, a pressure measured at the sensor.

64. The optical pressure sensor of claim 63, wherein the LED is a blue LED configured to emit blue light.

65. The optical pressure sensor of claim 63, wherein a spectrum of light emitted by the phosphor-based light filter has a peak wavelength in a range of 550-600 nm.

66. The optical pressure sensor of claim 65, wherein the spectrum has a peak wavelength in the range of 590-610 nm.
